(19) United States
(12) Patent Application Publication
(10) Pub. No.: US 2022/0361597 A1
(43) Pub. Date: Nov. 17, 2022

(54) APPAREL AND/OR METHODS FOR COMBATING VIRUSES

(71) Applicant: Larry W. Kiest, JR., Fort Lauderdale, FL (US)

(72) Inventor: Larry W. Kiest, JR., Fort Lauderdale, FL (US)

(21) Appl. No.: 17/662,264

(22) Filed: May 6, 2022

Related U.S. Application Data
(60) Provisional application No. 63/201,720, filed on May 11, 2021.

(51) Int. Cl. A41D 1/00; A62B 18/00; A62B 23/02; A61F 9/02; A61L 9/20; A41D 19/00

(52) U.S. Cl. CPC A41D 1/002; A62B 18/003; A62B 23/02; A61F 9/029; A61L 9/20; A41D 19/0024; A62B 18/082

(57) ABSTRACT

A device for creating an air curtain protecting a human respiratory system is described. The device can be a hat and/or collar worn by a person and includes a constant or on demand flow of air that passes across a person's face to create an air curtain. The flow of air moving across the face blows airborne viruses away from a person's eyes, nose, and mouth. Another concept includes a glove that is outfitted with a device that purifies the glove. A battery could be mounted on a person's wrist or forearm with a small wire running down the glove. Likewise, the glove could be outfitted with UV light embedded on the surface or just underneath a permeable/top layer of the glove. Still further, the glove can carry electrical pulses aimed to kill viruses every ten seconds or so.

### CROSS REFERENCE TO RELATED APPLICATIONS

This application claims priority under 35 U.S.C. § 119 to provisional patent application U.S. Ser. No. 63/201,720, filed May 11, 2021. The provisional patent application is herein incorporated by reference in its entirety, including without limitation, the specification, claims, and abstract, as well as any figures, tables, appendices, or drawings thereof.

### TECHNOLOGICAL FIELD

The present invention relates generally to an apparatus and/or corresponding method of use in at least the clothing industry. More particularly, but not exclusively, the present invention relates to hats and gloves for mitigating exposure to viruses.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

The coronavirus pandemic has severely impacted the livelihood of nations across the globe and brought many economies to a halt. The pandemic was caused by the spread of severe acute respiratory syndrome coronavirus 2 (SARS-CoV-2). The virus is spread primarily via small droplets from coughing, sneezing, and talking.

There are no proven vaccines or specific treatments for coronavirus 2019 (“COVID-19”). Management involves the treatment of symptoms, supportive care, isolation, and experimental measures.

Recommended preventive measures include hand washing, sanitizing a person's hands, covering mouth when coughing, social distancing, wearing a face mask in public, disinfecting surfaces, ventilating and air-filtering, and monitoring and self-isolation for people who suspect they may be infected. Authorities worldwide have responded by implementing travel restrictions, lockdowns, workplace hazard controls, and facility closures to slow the spread of the disease. Many places have also worked to increase testing capacity and trace contacts of the infected.

These measures have proven inadequate for many, including those who cannot isolate and cannot wear masks for medical reasons. For example, according to the Center for Disease Control (CDC), “cloth face coverings should not be placed on young children under age 2, anyone who has trouble breathing, or is unconscious, incapacitated, or otherwise unable to remove the mask without assistance.” Those persons are at increased risk for contracting the disease and can even be publicly shamed for their inability to wear a mask and/or self-isolate.

Regardless, people may also become infected by touching a contaminated surface and then touching their face. And, it is worth noting that while some devices have been created and sold that aid in sanitizing and/or disinfecting the user's hands, this can actually increase the user's risk of contracting COVID 19 because the user must contact or at least move toward devices which others have used relatively recently in order to utilize these devices. Moreover, these devices, like antigen tests and simple surgical masks, are quite susceptible to misuse and/or human error.

Thus, there exists a need in the art for alternative forms of apparel which similarly combat the spread of disease, and further, there exists a need in the art for apparel which is less susceptible to being worn in a manner which lowers the apparel's efficacy in combating spread of disease.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present invention to improve on or overcome the deficiencies in the art.

It is a further object, feature, and/or advantage of the present invention to provide comfort to the wearer, thereby encouraging the wearer to wear the apparel more frequently and discouraging the wearer from directly contacting his or her face with his or her hands.

It is still yet a further object, feature, and/or advantage of the present invention to create an air shield around the face of a user.

It is still yet a further object, feature, and/or advantage of the present invention to filter and/or purify air.

It is still yet a further object, feature, and/or advantage of the present invention to eliminate the need for a face masks proximate to the user's face. This can help persons having medical conditions which do not allow for the wearing of these types of face masks.

It is still yet a further object, feature, and/or advantage of the present invention to disinfect surfaces and/or objects known to transmit viruses.

It is still yet a further object, feature, and/or advantage of the present invention to combat and/or kill harmful bacteria.

It is still yet a further object, feature, and/or advantage of the present invention to utilize UV light to kill airborne COVID-19 particles and disinfect surfaces.

The apparel disclosed herein can be used in a wide variety of public locations which permit wearing hats and gloves. For example, the apparel can be worn in hospitals, subways, and other public locations.

It is preferred the apparatus be safe, cost effective, and durable. For example, the apparel can be adapted to resist thermal transfer and/or electric conductivity such that the apparel can be specially adapted for a wide variety of applications.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the present invention.

Methods can be practiced which facilitate use, manufacture, assembly, maintenance, and repair of apparel which accomplish some or all of the previously stated objectives.

Apparel can be incorporated into outfits and/or clothing kits which accomplish some or all of the previously stated objectives.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the present invention can be practiced are illustrated and described in detail, wherein like reference characters represent like components throughout the several views. The drawings are presented for exemplary purposes and may not be to scale unless otherwise indicated.

FIG. 1 shows a perspective view of the underside of a hat which can create an air shield for a wearer.

FIG. 2 shows a cross-sectional view of a glove which has wires and/or motors specially adapted to kill viruses.

FIG. 3 shows a partially hidden view of a holster and/or pocket which utilizes UV to purify gloves worn by the user.

FIG. 4 shows a shirt with a collar capable of creating an air shield for the wearer.

An artisan of ordinary skill need not view, within isolated figure(s), the near infinite number of distinct permutations of features described in the following detailed description to facilitate an understanding of the present invention.

### DETAILED DESCRIPTION

The present disclosure is not to be limited to that described herein. Mechanical, electrical, chemical, procedural, and/or other changes can be made without departing from the spirit and scope of the present invention. No features shown or described are essential to permit basic operation of the present invention unless otherwise indicated.

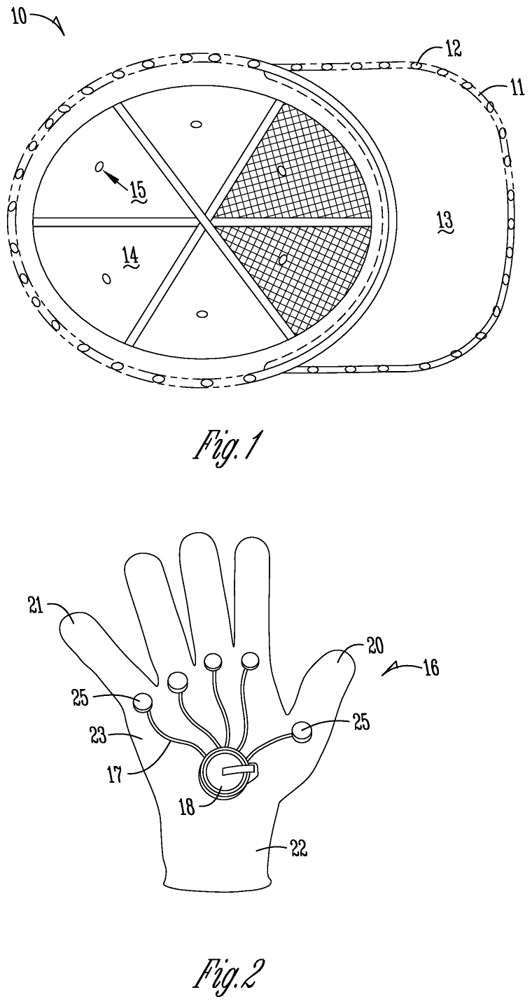

Referring now to the figures, apparel is shown in FIG. 1-4. More particularly, FIGS. 1-4 show a combination of apparel which can be worn together. The outfit includes a hat 10, a glove 16, a belt 26, a holster 30, and/or pocket 32, and shirt 38 with collar 46. The outfit allows the wearer to mitigate exposure to viruses and harmful bacteria during wear.

Apparel disclosed herein can be made from any suitable material(s), including cotton, wool, polyester, blends thereof, other suitable textiles, cellulosic fibres/viscose, bast fibres, leather, fur, artificial/synthetic materials specifically designed for wear by humans, and hardware (e.g. zippers, buttons, buckles, clasps, etc.). Moreover, the apparel disclosed herein can include any conceivable graphic designs located thereon or forming thereof so as to better appeal to each consumer's preference. The apparel can be adapted to be worn for specific climates (e.g. made to be worn in the summer or the winter), in certain social situations (e.g. specific to a wedding or a funeral), and/or be equipped with other functional aspects (e.g. include camouflage).

The apparel can be washable, reversible, easily maintained, and highly portable (e.g., easily transported as a combination from one location to the next). Alternatively, and/or in addition thereto, the apparel can be disposable and/or recyclable.

The hat 10 and shirt 38 when used alone or together can create a powerful air shield 40 to prevent outside air from coming into contact with a wearer's face before filtering. When used together, the same air source 44 supply both tubes 11, 42, thereby allowing air to flow therethrough. In some embodiments, one tube will use positive pressure and push air, while the air will use negative air and pull air to increase the strength of the air shield 40. In preferred embodiments, the tubes 11, 42 will range from about a one fourth (¼) inches to two (2) inches in diameter and can be made from a soft material such as textile, silicone, plastic, etc. The air source 44 can be a fan, blower, pump, hand pump, or constant stream.

The glove 16 and holster 30 can also work alone or in combination to help cause the death of viruses. The glove 16 and/or holster 30 can utilize ultrasonic waves, UV light, and/or electrical pulses to kill and/or disinfect viruses. Differentiating the means for killing and/or disinfecting the virus among the glove 16 and/or holster 30 can help increase the efficacy of the same.

Other various items which can be utilized by all of the apparel, such as batteries and/or an air source, can be attached to the belt 26. Switches to toggle power on and off to electrical systems worn by the user may also be located at the user's belt 26. Even sensors which sense other persons' presence may be included at the belt 26. Location of these components at the belt 26 however is not required, nor is inclusion of the belt 26.

In greater particularity, FIG. 1 will be appreciated to show at least a hat 10 comprising a bill 13, a crown 14, and eyelets 15. The hat 10 may take the shape of a baseball cap, as shown, or any other suitable and known shape for headwear. Where a baseball cap is used, the baseball cap may include a flex-fit (one-size-fits-all) or an adjustable strap. Also included on the cap 10 is an air tube 11 at a periphery of the bill 13. Orifices 12 in the air tube 11 permit air from an external air source to flow therethrough, provided the air tube 11 is operatively connected to an air source. The tube 11 may be held in place on the underside of the hat 10 with any suitable fastening means, including but not limited to adhesives, mechanical clips, or stiches sewn into the fabric of the hat 10.

The air shield caused by air flowing through the air tubes 11 shouldn't be so strong that it suffocates the wearer. Rather, it should more consistency direct air from one direction towards the user so the user can breathe and the air path more easily controlled/anticipated. Fresh air is ideally directed to the wearer for sustaining the user's breath, while contaminated air is deflected by the air tube 11 and/or air shield 40 (seen in FIG. 4).

Where the air flow path is known, the user may then employ a battery powered filter for combating airborne viruses may be used to help keep the user safe. In other words, the orifices 12 can be angled and/or oriented such that when the air blows therethrough, the air further shields the wearer from the airborne viruses in a predictable manner. In some embodiments, the orifices 12 are angled and/or oriented to push air outward and away from the wearer. In others, the orifices 12 are vertically angled and/or oriented to push air upward and/or downward from the wearer. In selected embodiments, the air tube 11 and orifices 12 may circumnavigate, or extend extends three hundred sixty degrees (360°) around a wearer's head, during wear.

In addition, the filter used with the air shield could take many forms. In some forms, the filter is a high efficiency particulate air (HEPA) filter. HEPA is a designation used to describe filters that are able to trap ninety-nine and ninety-seven hundredths percent (99.97%) of particles that are three tenths (0.3) microns. Most modern HEPA filters consist of interlaced glass fibers that are twisted and turned in myriad directions to create a fibrous maze. The filter could take many forms, including but not limited to: Direct Impaction: Large contaminants, such as certain types of dust, mold, and pollen, travel in a straight path, collide with a fiber, and stick to it. Sieving: The air stream carries a particle between two fibers, but the particle is larger than the gap, so it becomes ensnared. Interception: Airflow is nimble enough to reroute around fibers, but, thanks to inertia, particles continue on their path and stick to the sides of fibers. Diffusion: Small, ultrafine particles move more erratically than larger ones, so they're more likely to hit and stick to fibers. The filter could also be a carbon filter or the like, and can be combined with other types of filters. In any sense, the air used to blow away from a user could be filtered through a HEPA filter or other filtering member.

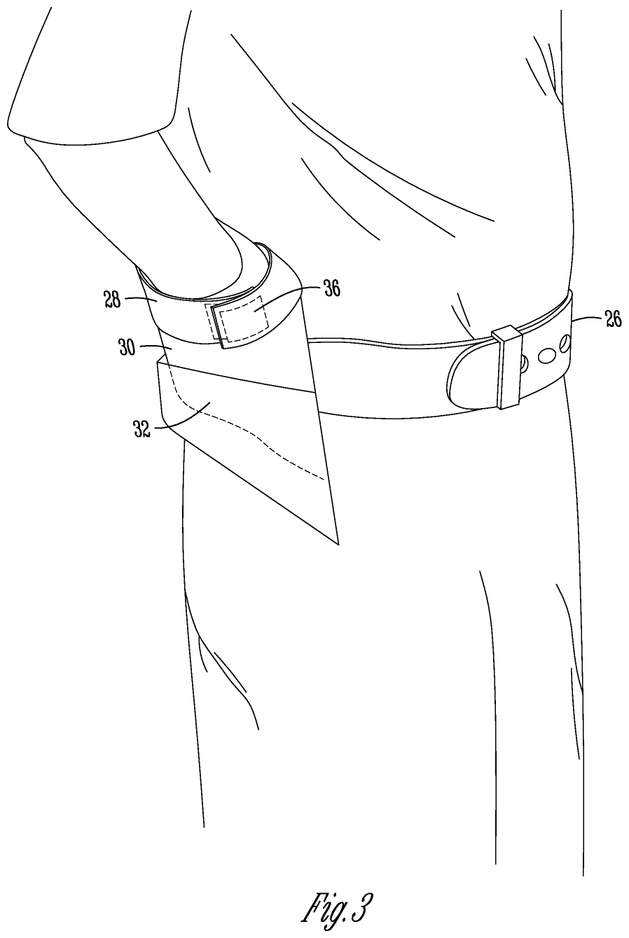

FIG. 2 will be appreciated to show a glove 16 comprising such mechanism(s) for killing viruses, which can be periodically activated by automatic timers associated with the battery 18. Such periodic timers can be turned on and off approximately every ten (10) seconds, however any suitable or preferable time period may be sued.

In the embodiments shown, vibration sources 25 capable of causing ultrasonic waves are located towards a distal portion of a wearer's palm 23. UV lights could also be located at this or any other suitable location. The UV lights could be very small lights embedded on the surface or just underneath a surface of the glove 16. The UV lights could also be located within a permeable layer or one of several layers of a glove 16. The UV light emitted from these light fixtures can be used to kill airborne COVID-19 particles, mold, other harmful particles containing DNA, and can further disinfect surfaces. Ultraviolet (UV) light kills cells by damaging their DNA because the UV initiates a reaction between two molecules of thymine, one of the bases that make up DNA.

Micro wires 17 can run throughout the glove 16, from wrist 22 through palm 23 to thumb 20 and fingers 21, to deliver electrical pulses that can also be used to kill viruses.

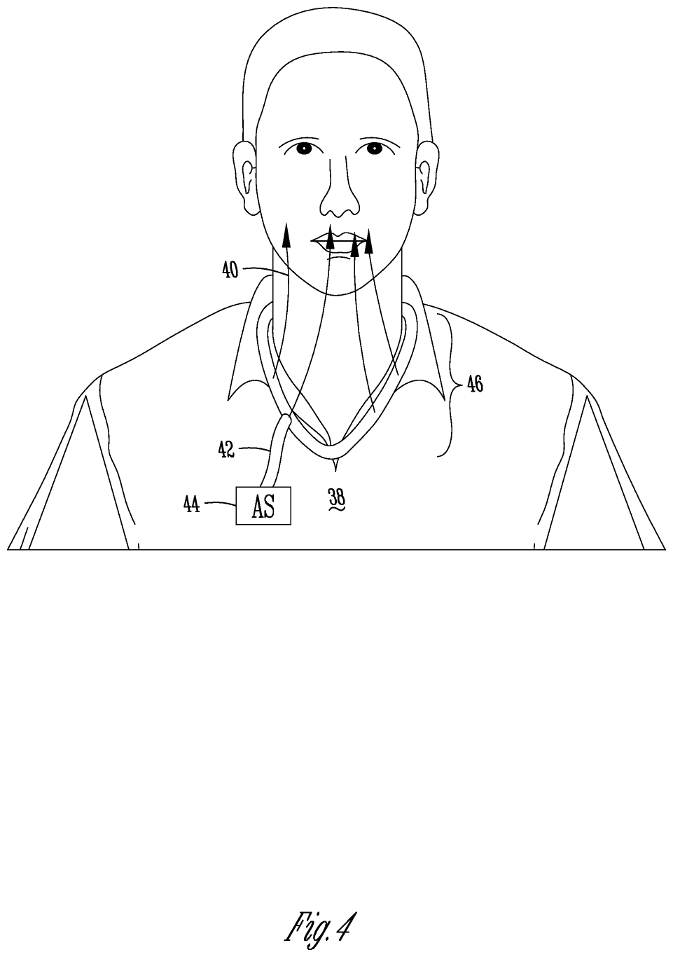

The glove 16 of FIG. 2 can also be placed in the pocket 32 or holster 30 of FIG. 3, before being secured by way of strap 28 and fastener 36 (e.g., hook and loop fastener). Within the pocket 32 or holster 30, there will exist similar mechanism(s) for killing viruses, which will preferably being automatically turned on only if a wearer inserts the glove 16 into the pocket 32 or holster 30. Again, differentiating the means for killing and/or disinfecting the virus among the glove 16 and/or holster 30 can help increase the efficacy of the same.

The pocket 32 and/or holster 30 can be outfitted on the wearer's apparel on one or both sides of same. The pocket 32 and/or holster 30 could be located on the left side one for a left-handed user and/or the right side for a right-handed user. Such a pocket 32 and/or holster 30 could be particularly beneficial if used in a workplace where the user regularly comes into contact with contaminated surface(s) and/or is already wearing a glove. For example, before a worker was to pick an object up they would first insert the glove(s) 16 into the pocket 32 and/or holster 30, purify the glove(s) 16, pick the object up knowing that the glove(s) 16 are clean, and move the object. After the object is set done, the user could again re-purify the glove(s) 16 again and even repeat the process as many time(s) as is desired by the wearer.

Finally, FIG. 4 shows the air tube 42 hidden within the wearer's shirt 38. In other words, the air tube 42 can be attached to the inside of a shirt or blouse, etc. The tube 42 may be held in place under the collar of the shirt with adhesives, mechanical clips, or sewn into the fabric of the shirt 38. The air is delivered via the tube 42 from the air source 44, which may be hidden under or within the shirt, worn on a belt 26, attached with an armband or secured in some way to the user. An air shield 40 may then be delivered up towards the nose and mouth of the user, if so desired.

Still other apparel can be created using at least some of the aspects described herein and may include scarves, hoodies, dresses, gowns, medical garments and gear, athletic apparel, sports equipment, personal protective gear, and/or any other suitable types of clothing.

Even when wearing the hat 10 and shirt 38 described herein, persons should take caution to cover their mouths and nose with a tissue when coughing or sneezing and/or the inside of the elbow if no tissue is available. Similar COVID-19 safe practices (e.g. social distancing) and protocols can enhance the effectiveness of the apparel described herein.

For example, in periods where persons are travelling outdoors and are located more than six feet apart, it is to be appreciated that exist embodiments where the user can manually idle or even stop use of the air shield 40. Still further, there can exist embodiments where sensors located within the apparel or at another location on the user's body automatically idle or stop use of the air shield 40 where the risk of transmission of a disease is negligible. These configurations can help save battery power over extended periods of time.

On the other hand, where transmission of a disease is more likely, the user could also manually increase, or a sensor could automatically increase, power to the air source 44 so as to create a more forceful air shield 40. Such configurations which allow for this feature can help prevent the wearer's immediate proximity from being overloaded with disease carrying particles, such as pathogens.

Thermal sensors and/or thermometers can also be employed to sense and/or monitor a body temperature, temperatures of air within the proximity of the user, temperatures of air within or external to the air shield, and even temperatures of electro-mechanical components of the apparel described herein, such as the air source 44. If a temperature rises above or below a predetermined level, the system could be configured to automatically adjust to bringing the temperature back to an acceptable level. This can help prevent the wearer and/or components such as the air source 44 from overheating.

Further, it is to be appreciated the hat 10 and shirt 38 can be worn with an additional layer(s) of protection for the facial region. Said additional layer can comprise eyeglasses, sunglasses, solid face shields, porous face shields, headbands, bandanas, other objects which lower the wearer's chances of contracting the coronavirus, and combinations thereof. The additional layer of protection is generally mechanical in nature and creates a barrier between the wearer and the environment, however in some embodiments can comprise chemicals applied to the user's face, similar to lotion or sunscreen. The additional layer of protection acts to protect the wearer from mucous membranes and splashes or sprays of bodily fluids emitted from other persons. These bodily fluids can comprise blood, perspiration, saliva, tears, and the like; and these bodily fluids are known to carry diseases other than just COVID-19.

Similarly, even when wearing the glove 16, proper hand hygiene after any cough or sneeze is encouraged. For example, persons may wash hands often with soap and water for at least twenty (20) seconds, especially when hands are visibly dirty and before eating. The CDC recommends using an alcohol-based hand sanitizer with at least sixty percent (60%) alcohol, but only when soap and water are not readily available.

Sanitizing of frequently touched surfaces is also recommended if keeping up businesses and/or public facilities; the United States Environmental Protection Agency maintains a list of products expected to be effective.

From the foregoing, it can be seen that the present invention accomplishes at least all of the stated objectives.

### GLOSSARY

Unless defined otherwise, all technical and scientific terms used above have the same meaning as commonly understood by one of ordinary skill in the art to which embodiments of the present invention pertain.

The terms “a,” “an,” and “the” include both singular and plural referents.

The term “or” is synonymous with “and/or” and means any one member or combination of members of a particular list.

The terms “invention” or “present invention” are not intended to refer to any single embodiment of the particular invention but encompass all possible embodiments as described in the specification and the claims.

As used herein, the term “exemplary” refers to an example, an instance, or an illustration, and does not indicate a most preferred embodiment unless otherwise stated.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

A “virus” is a submicroscopic infectious agent that replicates only inside the living cells of an organism. Viruses infect all types of life forms, from animals and plants to microorganisms, including bacteria and archaea.

“Bacteria” are a type of biological cell. Bacteria live in symbiotic and parasitic relationships with plants and animals. As used herein, “harmful” bacteria are those bacteria which are toxic and/or parasitic with respect to humans.

Ultraviolet (“UV”) light is a form of electromagnetic radiation with wavelength from 10 nm (with a corresponding frequency of approximately 30 PHz) to 400 nm (750 THz), shorter than that of visible light but longer than X-rays. UV radiation is present in sunlight. As used herein, UV light sources can include electric arcs and specialized lights, such as mercury-vapor lamps, tanning lamps, and black lights.

The “scope” of the present invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

What is claimed is:

1. A combination of apparel comprising:
a hat which utilizes an air shield to deflect viruses away from a wearer, said air shield comprising air passed through a filter;
an air source and/or vacuum to create the air shield; and
a shirt which aids in creating the air shield, wherein the shirt has a collar lined with a first tube and the hat has a bill with a second tube at its periphery, said first and second tubes having orifices that allow air to travel therethrough so as to create the air shield.

2. The combination of claim 1 further comprising an additional layer of protection for a facial region of the wearer, said additional layer of protection configured to protect the wearer from mucous membranes and splashes or sprays of body fluids from other persons.

3. The combination of claim 2 wherein the additional layer of protection comprises eyeglasses removably attached to said hat, and further wherein the eyeglasses are located between the air shield and the facial region.

4. The combination of claim 1 further comprising:
a belt the air source and/or vacuum attaches to; and
a glove which utilizes ultrasonic waves, UV light, and/or electrical pulses to kill and/or disinfect viruses.

5. The combination of claim 4 further comprising a holster attached to the belt which allows for the wearer to insert the glove so as to purify the glove of viruses.

6. The combination of claim 1 wherein the air source and/or vacuum is battery powered.

7. The combination of claim 1 wherein the air shield and any power to the glove can be toggled on and off

8. The combination of claim 7 wherein such toggling is automatic based upon whether a sensor senses presence of other persons within nearby proximity to the wearer.

9. A wearable device or apparel comprising:
a tube permitting air from an external air source to flow therethrough;
an operative connection to the external air source; the tube positioned to expel potentially contaminated air in front of a person's face by blowing filtered, clean air for combating airborne viruses.

10. The wearable device of claim 9 wherein the wearable device is a hat.

11. The wearable device of claim 10 wherein the hat comprises a bill and the tube is located at a periphery of the bill.

12. The wearable device of claim 10 wherein the wearable device is a necklace or is located at a collar of a shirt.

13. The wearable device of claim 9 further comprising orifices in the tube which permit air from an external air source to flow therethrough, said orifices being angled and/or oriented such that when the air blows therethrough, the air further shields the wearer from the airborne viruses.

14. The wearable device of claim 9 wherein the external air source utilizes negative pressure.

15. The wearable device of claim 9 wherein the tube extends 360° around a wearer's body during wear.

16. The wearable device of claim 9 wherein the filtered, clean air is provided by way of a HEPA filter.

17. A glove comprising:
a mechanism for killing viruses;
an automatic timer which periodically activates the mechanism; and
a battery which powers both the mechanism and the automatic timer.

18. The glove of claim 17 wherein the mechanism is an ultrasonic wave source.

19. The glove of claim 17 wherein the mechanism is a UV light.

20. The glove of claim 17 wherein the mechanism is micro wires which run throughout the glove.
